(19) United States
(12) Patent Application Publication
(10) Pub. No.: US 2023/0329245 A1
(43) Pub. Date: Oct. 19, 2023

(54) COMPOSITIONS AND METHODS OF USE THEREOF

(71) Applicant: Curie Co. Inc., Durham, NC (US)

(72) Inventors: Erika A. MILCZEK, Durham, NC (US); Will SHINDEL, Durham, NC (US)

(21) Appl. No.: 18/044,226

(22) PCT Filed: Sep. 7, 2021
(86) PCT No.: PCT/US2021/049326

§ 371 Date: Mar. 7, 2023

Related U.S. Application Data
(60) Provisional application No. 63/075,763, filed on Sep. 8, 2020.

(51) Int. Cl. A01N 63/50; C12N 9/10; A01P 1/00; C12N 15/63

(52) U.S. Cl. CPC A01N 63/50; C12N 9/1044; A01P 1/00; C12N 15/63

(57) ABSTRACT

Enzyme-based compositions for broad spectrum microbial control and/or preservation are disclosed herein. The disclosed compositions include crosslinking enzymes, optionally, in the form of a zymogen, and include antimicrobial peptides, proteins, polymers, and optionally may include chemical preservatives.

### CROSS-REFERENCE TO RELATED APPLICATIONS

This application claims the benefit of U.S. Provisional Application No. 63/075,763, filed Sep. 8, 2020, which is incorporated herein by reference in its entirety.

### STATEMENT REGARDING FEDERALLY SPONSORED RESEARCH

This invention was partially made with government support under Grant No. 2026057, awarded by the National Science Foundation. The government has certain rights in this invention.

### INCORPORATION BY REFERENCE

The sequence listing provided in the file named XXXXX with a size of XXKB which was created on XXXXXX, and which is filed herewith, is incorporated by reference in its entirety.

### FIELD

The field pertains to active crosslinking enzymes and formulations thereof for use as a preservative and/or antimicrobial agent.

### BACKGROUND

Preservative compositions for protecting and preserving formulations against bacterial or fungal attack are known in the art and have a wide variety of applications in fields such as personal care products, household and industrial products, health and hygiene products, and pharmaceuticals. Conventional preservative blends have included traditional active ingredients, such as formaldehyde, formaldehyde-releasers, phenolic compounds, quaternary ammonium compounds, halogenated compounds, and/or parabens, due to the good bacterial and fungicidal properties achieved by these types of compounds (U.S. Pat. No. 9,661,847 B2). In addition to chemicals and small molecules, biocidal enzymes and proteins have been used as biocompatible preservatives in the food (Malhotra, et al. (2015) Frontiers in Microbiology 6:611), healthcare (Kaplan, et al. (2010) Journal of Dental Research 89:205-218), and marine (Olsen, et al. (2007) Biofouling 23:369-383) industries. Examples of these enzymes include: oxidases and peroxidases (e.g., glucose oxidase, laccase), which generate oxidizing species for biocidal activity; lytic enzymes, including proteases, hydrolases, and lyases (e.g., lysozyme, lysostaphin, subtilisin, amylase, cellulase, chitinase, lipase), which degrade the surface of microbes (e.g., fungi, viruses, bacteria); nucleases (e.g., lactoferrin, DNase, RNase), which hydrolyze nucleic acids, such as RNA or DNA; and antimicrobial peptides (e.g., nisin, pediocin), which kill microbes by creating pores in the cell wall, resulting in cell rupture and leakage of cell contents. Additive effects between antimicrobials in preservative blends not only allow for lowering the concentration of individual ingredients, but also hinder antimicrobial resistance because organisms are attacked by multiple modes of action, providing broad spectrum antimicrobial action.

Enzyme based antimicrobial compositions have been identified by others. For example, U.S. Pat. No. 5,326,561 discloses an antifungal composition using lytic enzymes, such as chitinolytic enzymes, glucanolytic enzymes and cellulases. However, use of lytic enzymes may be problematic, since such enzymes can destroy consumer product formulations that contain: (a) esters, which are used as conditioners and shine increasing agents, (b) proteins (e.g., keratin and peptide hair/skin conditions), and/or (c) carbohydrates (e.g., gums and other thickeners). Accordingly, there remains a need for agents having antimicrobial (e.g., bactericidal and fungicidal) activity without deleterious side effects

Alkylators and crosslinking chemical agents have been successfully employed for broad spectrum microbial control. Chief among these are aldehyde-based biocides, such as formaldehyde and glutaraldehyde, which have been known for years to have broad spectrum antimicrobial activity. It is well known that these aldehydes crosslink vital cellular components, such as proteins, enzymes, and nucleic acids, which are needed for cellular function. This action results in inhibition of microbial growth or cell death. However, the reactive nature of aldehydes means they may decompose quickly within a formulation through undesired chemical reaction. Additionally, formaldehyde is classified as Category 3 CMR (carcinogenic, mutagenic, and reproductive toxicity).

A few antimicrobials that slowly release formaldehyde are still being used and commercially manufactured. Due to the paucity of effective and well accepted antimicrobials, the industry is forced to continue using formaldehyde donors like DMDM hydantoin, imidazolidinyl urea, and diazolidinyl urea. The formaldehyde released by these substances is capable of reacting with several cosmetic ingredients via its reactive aldehydic functionality. For example, the only available and globally approved UV-A absorber, Avobenzone, reacts with formaldehyde that is released by formaldehyde derivatives. This is a disadvantage for sunscreen formulations.

PCT Publication No. WO 2020/181099, having International Publication Date Sep. 10, 2020, discloses antimicrobial compositions which may have a crosslinking enzyme either in active form or in the form of a zymogen, wherein such compositions may be used to improve shelf-life of a product.

The present disclosure illustrates an alternative to aldehyde-based, crosslinking chemical preservatives. Specifically, this disclosure provides an enzyme-based mechanism through the use of crosslinking enzymes for microbial control. Crosslinking enzymes are highly precise for certain functional groups or peptide sequences, allowing for compatibility with chemical preservatives or biological based antimicrobials (e.g., peptides, proteins, and enzymes). The enzymes presented herein are larger than 10 kDa in molecular weight, meaning there is low risk of skin penetration. Additionally, the enzymes presented herein are highly specific for crosslinking amino acid residues without reacting with nucleic acids, alleviating key concerns of safety, and of mutagenic and carcinogenic properties associated with chemical crosslinking agents like formaldehyde. Crosslinking enzymes provide a green, sustainable alternative to chemical crosslinking agents for broad spectrum microbial control.

### SUMMARY

In a first embodiment, there is disclosed a composition comprising (a) at least one crosslinking enzyme, optionally in the form of a zymogen, in combination with (b) at least one component selected from the group consisting of enzymes, peptides, and/or proteins, optionally having antimicrobial activity, and optionally further in combination with (c) at least one chemical preservative, wherein the composition comprising (a) in combination with (b), and optionally further in combination with (c), has at least one activity selected from the group consisting of preservative and antimicrobial.

In a second embodiment, the at least one crosslinking enzyme is selected from the group consisting of transglutaminases, lysyl oxidases, tyrosinases, laccases, sortases, formylglycine-generating enzymes, and sulfhydryl oxidases. Preferably, the at least one crosslinking enzyme is a transglutaminase. Most preferably, the at least one crosslinking enzyme has at least 90% sequence identity with the amino acid sequence set forth in SEQ ID NO:2.

In a third embodiment, there is disclosed that the at least one component, of any of the embodiments described herein, having antimicrobial activity is selected from the group consisting of lysozymes, chitinases, lipases, lysins, lysostaphins, glucanases, DNases, RNases, lactoferrins, glucose oxidases, peroxidases, lactoperoxidases, lactonases, acylases, dispersin B, amylases, proteases, cellulases, nisin, bacteriocins, siderophores, polymyxins, and defensins.

In a fourth embodiment, there is disclosed that the at least one chemical preservative, of any of the embodiments described herein, is selected from the group consisting of quaternary ammonium compounds, detergents, chaotropic agents, organic acids, alcohols, glycols, aldehydes, oxidizers, parabens, isothiazolinones, and cationic polymers.

In a fifth embodiment, there is disclosed that the (a) at least one crosslinking enzyme is in the form of a zymogen and the (b) at least one component comprises an enzyme, further wherein the zymogen and the enzyme interact to produce an active enzyme having at least one activity selected from the group consisting of preservative and antimicrobial.

In a sixth embodiment, there is disclosed an expression vector comprising at least one heterologous nucleic acid sequence that encodes at least one crosslinking enzyme, optionally in the form of a zymogen, wherein said heterologous nucleic acid sequence is optionally operably linked to at least one regulatory sequence, and wherein the expression vector is capable of transforming a host cell to express, either intracellularly or extracellularly, at least one crosslinking enzyme so that the transformed host cell is inactivated, inhibited, or killed. Additionally, the at least one crosslinking enzyme is selected from the group consisting of transglutaminases, lysyl oxidases, tyrosinases, laccases, sortases, formylglycine-generating enzymes, and sulfhydryl oxidases. Preferably, the at least one crosslinking enzyme is a transglutaminase. Most preferably, the at least one crosslinking enzyme has at least 90% sequence identity with the amino acid sequence set forth in SEQ ID NO:2.

### BRIEF DESCRIPTION OF THE SEQUENCES

SEQ ID NO:1 corresponds to the transglutaminase (Tgase) sequence of Streptomyces mobaraensis mature form.

SEQ ID NO:2 corresponds to a variant of the sequence of SEQ ID NO:1 mature form.

SEQ ID NO:3 corresponds to a sequence of a variant of Streptomyces mobaraensis Tgase zymogen form (pro-Tgase).

SEQ ID NO:4 corresponds to the wild-type Pro-TAMEP sequence of Streptomyces mobaraensis zymogen form (pro-TAMEP; UniProt P83543) containing a C-terminal hexa-His-tag.

SEQ ID NO:5 corresponds to the wild-type Pro-SM-TAP sequence of Streptomyces mobaraensis zymogen form (pro-SM-TAP; UniProt P83615) containing a C-terminal hexa-His-tag.

SEQ ID NO:6 corresponds to the variant transglutaminase sequence of Streptomyces mobaraensis in SEQ ID NO:2, mature form, including N-terminal Methionine and C-terminal peptide linker and hexa-His-Tag.

### DETAILED DESCRIPTION

All patents, patent applications, and publications cited herein are incorporated by reference in their entireties.

In this disclosure, many terms and abbreviations are used. The following definitions apply unless specifically stated otherwise.

As used herein, the singular forms “a,” “an,” and “the” include plural references unless the context clearly dictates otherwise. For example, the term “a compound” or “at least one compound” may include a plurality of compounds, including mixtures thereof. The terms “a,” “an,” “the,” “one or more,” and “at least one,” for example, can be used interchangeably herein.

The terms “and/or” and “or” are used interchangeably herein and refer to a specific disclosure of each of the two specified features or components with or without the other. Thus, the term “and/or” as used in a phrase such “A and/or B” herein is intended to include “A and B,” “A or B,” “A” (alone), and “B” (alone). Likewise, the term “and/or” as used a phrase such as “A, B and/or C” is intended to encompass each of the following aspects: A, B and C; A, B or C; A or C; A or B; B or C; A and C; A and B; B and C; A (alone); B (alone); and C (alone).

Words using the singular include the plural, and vice versa, unless the context clearly dictates otherwise.

The terms “comprises,” “comprising,” “includes,” “including,” “having” and their conjugates are used interchangeably and mean “including but not limited to.” It is understood that wherever aspects are described herein with the language “comprising,” otherwise analogous aspects described in terms of “consisting of” and/or “consisting essentially of” are also provided.

The term “consisting of” means “including and limited to.”

The term “consisting essentially of” means the specified material of a composition, or the specified steps of a methods, and those additional materials or steps that do not materially affect the basic characteristics of the material or method.

Throughout this application, various embodiments can be presented in a range format. It should be understood that the description in range format is merely for convenience and brevity and should not be construed as an inflexible limitation on the scope of the embodiments described herein. Accordingly, the description of a range should be considered to have specifically disclosed all the possible subranges as well as individual numerical values within that range. For example, description of a range, such as from 1 to 6 should be considered to have subranges such as from 1 to 2, from 1 to 3, from 1 to 4 and from 1 to 5, from 2 to 3, from 2 to 4, from 2 to 5, from 2 to 6, from 3 to 4, from 3 to 5, from 3 to 6, etc., as well as individual numbers within that range, for example, 1, 2, 3, 4, 5 and 6. This applies regardless of the breadth of the range.

The term “about” as used herein can allow for a degree of variability in a value or range, for example, within 10%, within 5%, or within 1% of a stated value or of a stated limit of a range.

The terms “peptides”, “proteins” and “polypeptides” are used interchangeably herein and refer to a polymer of amino acids joined together by peptide bonds. A “protein” or “polypeptide” comprises a polymeric sequence of amino acid residues. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code. Mutations can be named by the one letter code for the parent amino acid, followed by a position number and then the one letter code for the variant amino acid. For example, mutating glycine (G) at position 87 to serine (S) is represented as “G087S” or “G87S”. When describing modifications, a position followed by amino acids listed in parentheses indicates a list of substitutions at that position by any of the listed amino acids. For example, 6(L, I) means position 6 can be substituted with a leucine or isoleucine. At times, in a sequence, a slash (/) is used to define substitutions, e.g. F/V, indicates that the position may have a phenylalanine or valine at that position.

The term “crosslinking enzyme” refers to an enzyme that catalyzes a reaction between a functional group of an amino acid residue of a protein or polypeptide such as the amide functional groups of glutamine or asparagine, the amine group of a lysine, or the phenolic functional group of a tyrosine, with (a) a different reactive functional group of a protein or polypeptide amino acid residue, e.g., the amine functional group of a lysine, the hydroxyl group of a serine, or the phenolic hydroxyl group of a tyrosine, either by intermolecular or intramolecular reactions, or with (b) a reactive functional group of a molecule or substance of interest. One example of a “crosslinking enzyme” is transglutaminase (Tgase, EC2.3.2.13) that catalyzes the formation of an isopeptide bond between a primary amine, for example the epsilon-amine of a lysine molecule, and the acyl group of a protein- or peptide-bound glutamine. A second example of a “crosslinking enzyme” is tyrosinase (EC 1.14.18.1), a copper-containing oxidase that oxidizes phenols such as tyrosine and dopamine to form reactive o-quinones which readily form crosslinks with solvent-exposed lysyl, tyrosyl, and cysteinyl residues, as well as numerous small molecules. A third example of a “crosslinking enzyme” is laccase, a multi-copper oxidase found in plants, fungi and bacteria, that oxidizes phenolic substrates performing one-electron oxidations, resulting in crosslinking. A fourth example of a “crosslinking enzyme” is lysyl oxidase, a copper dependent oxidase that catalyzes the conversion of lysine molecules into reactive aldehydes that form crosslinks with other proteins and peptides as well as numerous small molecules.

The terms “signal sequence” and “signal peptide” refer to a sequence of amino acid residues that may participate in the secretion or direct transport of the mature or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The terms “zymogen” and “proenzyme” are used interchangeably herein and refer to an inactive precursor of an enzyme, which may be converted into an active or mature enzyme by catalytic action, such as via proteolytic cleavage of a pro-sequence.

The term “mature or active” form of a protein, polypeptide, or peptide refers to the functional form of the protein, polypeptide, or enzyme without a signal, silencing, or chaperoning propeptide sequence. Additionally, the mature enzyme may be truncated relative to the mature sequence while maintaining the desired activity (e.g., antimicrobial and/or preservative).

The term “wild-type” in reference to an amino acid sequence or nucleic acid sequence indicates that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant/engineered nucleic acids and protein sequences produced in the laboratory or modification of the wild-type sequence).

The term “derived from” encompasses the terms “originated from,” “obtained from,” “obtainable from,” “isolated from,” “purified from,” and “created from,” and generally indicates that one specified material finds its origin in another specified material or has features that can be described with reference to another specified material.

The terms “isolated,” “purified,” “separated,” and “recovered” as used herein refer to a material (e.g., a protein, nucleic acid, or cell) that is removed from at least one component with which it is naturally associated. For example, these terms may refer to a material which is substantially or essentially free from components which normally accompany it as found in its native state, such as, for example, an intact biological system. An isolated nucleic acid molecule includes a nucleic acid molecule contained in cells that ordinarily express the nucleic acid molecule, but the nucleic acid molecule is present extrachromosomally or at a chromosomal location that is different from its natural chromosomal location.

As used herein with regard to amino acid residue positions, “corresponding to” or “corresponds to” or “correspond to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related protein or a reference protein.

The term “amino acid” refers to the basic chemical structural unit of a protein, peptide, or polypeptide. The following abbreviations used herein to identify specific amino acids can be found in Table 1.

TABLE 1
One and Three Letter Amino Acid Abbreviations

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Thermostable serine acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid or as defined herein | Xaa | X |

One of ordinary skill in the art will appreciate that modifications of amino acid sequences disclosed herein can be made while retaining the function associated with the disclosed amino acid sequences. For example, it is well known in the art that alterations in a gene which result in the production of a chemically equivalent amino acid at a given site, but do not affect the functional properties of the encoded protein, are common.

The term “mutation” herein refers to a change introduced into a parental sequence, including, but not limited to, substitutions, insertions, and deletions (including truncations), thereby producing a “mutant.” The consequences of a mutation include, but are not limited to, the creation of a new character, property, function, phenotype or trait not found in the protein encoded by the parental sequence.

Related (and derivative) proteins encompass “variant” or “mutant” proteins, which terms are used interchangeably herein. Variant proteins differ from another (i.e., parental) protein and/or from one another by a small number of amino acid residues. A variant may include one or more amino acid mutations (e.g., amino acid deletion, insertion or substitution) as compared to the parental protein from which it is derived. Alternatively or additionally, variants may have a specified degree of sequence identity with a reference protein or nucleic acid, e.g., as determined using a sequence alignment tool, such as BLAST, ALIGN, and CLUSTAL. For example, variant proteins or nucleic acid may have at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% amino acid sequence identity with a reference sequence and integer percentage therebetween.

The term “codon optimized”, as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes.

The term “gene” refers to a nucleic acid molecule that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature Accordingly, a chimeric gene may comprise regulatory sequences and coding sequences that are derived from different sources, or regulatory sequences and coding sequences derived from the same source, but arranged in a manner different from that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “coding sequence” refers to a nucleotide sequence which codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding sites, and stem-loop structures.

The term “operably linked” refers to the association of nucleic acid sequences on a single nucleic acid molecule so that the function of one is affected by the other. For example, a promoter is operably linked with a coding sequence when it is capable of affecting the expression of that coding sequence, i.e., the coding sequence is under the transcriptional control of the promoter. Coding sequences can be operably linked to regulatory sequences in sense or antisense orientation.

The terms “regulatory sequence” or “control sequence” are used interchangeably herein and refer to a segment of a nucleotide sequence which is capable of increasing or decreasing expression of specific genes within an organism. Examples of regulatory sequences include, but are not limited to, promoters, signal sequence, operators, and the like. As noted above, regulatory sequences can be operably linked in sense or antisense orientation to the coding sequence/gene of interest.

“Promoter” or “promoter sequences” refer to a regulatory sequence that is involved in binding RNA polymerase to initiate transcription of a gene. The promoter may be an inducible promoter or a constitutive promoter.

The “3′ non-coding sequences” refer to DNA sequences located downstream of a coding sequence and include sequences encoding regulatory signals capable of affecting mRNA processing or gene expression, such as termination of transcription.

The term “transformation” as used herein refers to the transfer or introduction of a nucleic acid molecule into a host organism. The nucleic acid molecule may be introduced as a linear or circular form of DNA The nucleic acid molecule may be a plasmid that replicates autonomously, or it may integrate into the genome of a production host. Hosts containing the transformed nucleic acid are referred to as “transformed” or “recombinant” or “transgenic” organisms or “transformants”.

The terms “recombinant” and “engineered” refer to an artificial combination of two otherwise separated segments of nucleic acid sequences, e.g., by chemical synthesis or by the manipulation of isolated segments of nucleic acids by genetic engineering techniques. For example, DNA in which one or more segments or genes have been inserted, either naturally or by laboratory manipulation, from a different molecule, from another part of the same molecule, or from an artificial sequence, results in the introduction of a new sequence in a gene and subsequently in an organism. The terms “recombinant”, “transgenic”, “transformed”, “engineered”, “genetically engineered” and “modified for exogenous gene expression” are used interchangeably herein.

The term “vector” refers to a polynucleotide sequence designed to introduce nucleic acids into one or more cell types. Vectors include, but are not limited to, cloning vectors, expression vectors, shuttle vectors, plasmids, phage particles, bacteriophages, cassettes and the like.

An “expression vector” as used herein means a DNA construct comprising a DNA sequence which is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “expression”, as used herein, refers to the production of a functional end-product (e.g., an mRNA or a protein) in either precursor or mature form. Expression may also refer to translation of mRNA into a polypeptide.

Expression of a gene involves transcription of the gene and translation of the mRNA into a precursor or mature protein.

“Mature” protein refers to a post-translationally processed polypeptide, i.e., one from which any signal sequence, pre- or propeptides present in the primary translation product have been removed.

“Precursor” protein refers to the primary product of translation of mRNA; i.e., with pre- and propeptides still present. Pre- and propeptides may be but are not limited to intracellular localization signals.

“Stable transformation” refers to the transfer of a nucleic acid fragment into a genome of a host organism, including both nuclear and organellar genomes, resulting in genetically stable inheritance.

In contrast, “transient transformation” refers to the transfer of a nucleic acid fragment into the nucleus, or DNA-containing organelle, of a host organism resulting in gene expression without integration or stable inheritance.

The terms “recombinant construct,” “expression construct,” “recombinant expression construct” and “expression cassette” are used interchangeably herein. A recombinant construct comprises an artificial combination of nucleic acid fragments, e.g., regulatory and coding sequences that are not all found together in nature. For example, a construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened to obtain cell lines displaying the desired expression level and pattern. Such screening may be accomplished using standard molecular biological, biochemical, and other assays, including Southern analysis of DNA, Northern analysis of mRNA expression, polymerase chain reaction (PCR), real time quantitative PCR (qPCR), reverse transcription PCR (RT-PCR), immunoblotting analysis of protein expression, enzyme, or activity assays, and/or phenotypic analysis.

The terms “host” and “host cell” are used interchangeably herein and refer to any prokaryotic cells or eukaryotic cells, such as a plant, organism, or cell of any plant or organism, whether human or non-human, into which a recombinant construct can be stably or transiently introduced to express a gene. This term encompasses any progeny of a parent cell, which is not identical to the parent cell due to mutations that occur during propagation.

The term “antimicrobial” refers to any agent or combination of agents which is intended to kill, inactivate or inhibit the growth of any microbes such as bacteria, fungi, viruses, yeast, mold, and the like. The terms “antimicrobial” and “biocidal” are used interchangeably herein.

The term “broad spectrum antimicrobial” is one that acts against a wide range of microorganisms, for example Gram-positive bacteria, Gram-negative bacteria, yeast, mold, viruses, etc.

The term “potentiate” refers to making effective or active or more effective or active.

The terms “microorganism” and “microbe” are used interchangeably herein and refer to living thing that is so small that it can only be seen with a microscope., i.e., a microscopic organism. Microbes may exist in a single-celled form or in a colony of cells or in a biofilm. Microbes include eukaryotes and prokaryotes such as bacteria, archaea, protozoa, fungi, algae, amoebas, viruses and the like.

As used herein, the term “product” is intended to refer to a preparation, composition, or article of manufacture that has a specific utility that may require preservation or use of the antimicrobial enzyme composition as described herein, such as a consumer packaged goods. Examples include, but are not limited to, personal care products, household products, cosmetics, over-the-counter therapeutics, pharmaceutical preparations, paints, coatings, adhesives, food, and formulations for purchase by a consumer.

The term “composition” refers to a combination of two or more substances, including an enzyme (e.g., preservative and/or antimicrobial) composition as described herein.

“Effective amount” as used herein refers to an amount (e.g., minimum inhibitory concentration (MIC)) of a preservative composition as disclosed herein that is sufficient to prevent or inhibit microbial growth. The preservative compositions described herein may be active against Gram-positive bacteria, Gram-negative bacteria, yeast, fungi, and/or molds.

The term “pathogen” refers to any organism or substance that is capable of causing disease. Examples of disease-causing organisms, include but are not limited to, bacteria, fungi, viruses, protozoa and parasites.

“Pharmaceutically acceptable” means approved by a regulatory agency of the Federal or a state government or listed in the U.S. Pharmacopoeia or other generally recognized pharmacopoeia for use in animals and, more particularly, in humans.

“Pharmaceutically acceptable vehicle” or “pharmaceutically acceptable excipient” refers to any diluent, adjuvant, excipient or carrier with which an expression vector or antimicrobial composition as described herein may be administered.

The term “preservative” refers to a substance or agent that is added to a product to prevent decomposition by microbial growth or by undesirable chemical changes. Also included in “preservatives” are antioxidants and oxygen removal substances. Examples of such antioxidants and oxygen removal substances include, but are not limited to, ascorbic acid, superoxide dismutase, catalase and the like. Examples of products to which preservatives may be added include, but are not limited to, food products, beverages, pharmaceutical drugs, paints, biological samples, cosmetics, wood, household cleaning products, personal care products and the like.

“Optional” or “optionally” means that the subsequently described event, circumstance, or material may or may not occur or be present, and that the description includes instances where the event, circumstance, or material occurs or is present and instances where it does not occur or is not present.

The term “shelf life” refers to the length of time for which an item (e.g., a product as described herein) remains usable, fit for consumption, or saleable.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated.

In a first embodiment, there is disclosed a composition comprising (a) at least one crosslinking enzyme, optionally in the form of a zymogen, in combination with (b) at least one component selected from the group consisting of enzymes, peptides, or proteins, optionally having antimicrobial activity, and optionally in further combination with (c) at least one chemical preservative, wherein the composition comprising (a) in combination with (b) and optionally in further combination with (c), has at least one activity selected from the group consisting of preservative and antimicrobial.

Examples of suitable crosslinking enzymes that maybe used herein include, but are not limited to, transglutaminases, lysyl oxidases, tyrosinases, laccases, sortases, formylglycine-generating enzymes, and sulfhydryl oxidases.

As is noted above, “crosslinking” refers to an enzyme that catalyzes a reaction between a functional group of an amino acid residue of a protein or polypeptide, such as the amide functional group of a glutamine or asparagine, the amine group of a lysine, or the phenolic functional group of a tyrosine, with (a) a different reactive functional group of a protein or polypeptide amino acid residue, e.g., the amine functional group of a lysine, the hydroxyl group of a serine, or the phenolic hydroxyl group of a tyrosine, either by intermolecular or intramolecular reactions, or with (b) a reactive functional group of a molecule or substance of interest. One example of a “crosslinking enzyme” is transglutaminase (Tgase, EC 2.3.2.13) that catalyzes the formation of an isopeptide bond between a primary amine, for example the epsilon-amine of a lysine molecule, and the acyl group of a protein- or peptide-bound glutamine. A second example of a “crosslinking enzyme” is tyrosinase (EC 1.14.18.1) a copper-containing oxidase that oxidizes phenols such as tyrosine and dopamine to form reactive o-quinones, which readily forms crosslinks with solvent-exposed lysyl, tyrosyl, and cysteinyl residues, as well as numerous small molecules. A third example of a “crosslinking enzyme” is laccase, a multi-copper oxidase found in plants, fungi and bacteria, that oxidizes phenolic substrates performing one-electron oxidations, resulting in crosslinking. A fourth example of a “crosslinking enzyme” is lysyl oxidase, a copper dependent oxidase that catalyzes the conversion of lysine molecules into highly reactive aldehydes that crosslink with other proteins and peptides as well as numerous small molecules.

In some embodiments, the compositions include at least one crosslinking enzyme, e.g., comprising or consisting essentially of at least one crosslinking enzyme, in an amount effective to inhibit microbial (e.g., bacterial, fungal) growth, e.g., inhibition of 50% to 100%, or any of at least about 50%, 51%, 52%, 53%, 54%, 55%, 56%, 57%, 58%, 59%, 60%, 61%, 62%, 63%, 64%, 65%, 66%. 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of microbial growth, in a product to be preserved.

Preferably, the crosslinking enzymes can be selected from transglutaminases, lysyl oxidases, and tyrosinases, which usually exhibit cellular toxicity in an active enzyme form.

Most preferably, the crosslinking enzyme, includes, but is not limited to, transglutaminases, e.g., Streptomyces mobaraensis transglutaminase (SEQ ID NO:1), or a variant thereof (e.g., SEQ ID NO:2). Most specifically, the crosslinking enzyme has at least 90% sequence identity with the amino acid sequence set forth in SEQ ID NO:2.

In some embodiments, the enzyme is a crosslinking enzyme, such as, but not limited to, a transglutaminase, e.g., Streptomyces mobaraensis transglutaminase (SEQ ID NO:1), or a variant thereof (e.g., SEQ ID NO:2), having antimicrobial and/or preservative activity and at least at least about 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence set forth in SEQ ID NO:2.

A transglutaminase (Tgase, EC2.3.2.13) is an enzyme that catalyzes the formation of an isopeptide bond between a primary amine, for example, the ε-amine of a lysine molecule, and the acyl group of a protein- or peptide-bound glutamine. Transglutaminases may catalyze a transamidation reaction between glutamyl and lysyl side chains of target proteins. Proteins possessing Tgase activity have been found in microorganisms, plants, invertebrates, amphibians, fish and birds. In contrast to eukaryotic Tgases, Tgases of microbial origin are calcium-independent, which represents a major advantage for their practical use.

In some embodiments, the transglutaminase is a microbial transglutaminase, for example the Ca²⁺-independent microbial transglutaminase (Tgase) of a variant of Streptomyces mobaraensis. In some particularly preferred embodiments, the Tgase is a microbial Tgase and preferably is the Ca²⁺-independent microbial transglutaminase (Tgase) of a variant of Streptomyces mobaraensis. In some particularly preferred embodiments, the Tgase is a more stable mutational variant of Streptomyces mobaraensis Tgase, such as SEQ ID NO:2. Well defined microbial Tgases are shown in Table 2, reproduced from Zhang, et al. (2010) Biotechnol. Genet. Eng. Rev. 26:205-222, with additions from Steffen, et al. (2017) J. Biol. Chem. 292(38):15622-15635.

Transglutaminase belongs to the transferase class of enzymes (Heck et al. (2013) Applied Microbiology and Biotechnology 97:461-475). Transferases catalyze the transfer of functional groups such as methyl, hydroxymethyl, formal, glycosyl, acyl, alkyl, phosphate, and sulfate groups by means of a nucleophilic substitution reaction. Transferases can be divided into ten categories, based on the group(s) transferred. The different groups transferred include single-carbon groups, aldehyde or ketone groups, acyl groups or groups that become alkyl groups during transfer, glycosyl groups, as well as hexoses and pentoses, alkyl or aryl groups, other than methyl groups, nitrogenous groups, and phosphorus-containing groups; subclasses are based on the acceptor (e.g. alcohol, carboxyl, etc.), sulfur-containing groups, selenium-containing groups, and molybdenum or tungsten.

TABLE 2
Well Defined Microbial Tgases

| Year | Strain | Focus of the development |
|---|---|---|
| 1989 | Streptoverticillium | Strain isolation |
| | mobaraense | |
| 1996 | Streptoverticillium | Substrate optimization |
| | mobaraense | |
| 1997 | Streptoverticillium | Substrate optimization |
| | cinnamoneum | |
| 1998 | Streptoverticillium | Metabolic optimization |
| | mobaraense | |
| 2000 | Actinomadura sp. | Strain isolation |
| 2001 | Streptoverticillium | Environmental control strategies |
| | mobaraense | |
| 2002 | Streptoverticillium | Environmental control strategies |
| | mobaraense | |
| 2002 | Streptoverticillium | Environmental control strategies |
| | mobaraense | |
| 2004 | Streptoverticillium | Strain isolation |
| | ladakanum | |
| 2004 | Streptoverticisllium | Substrate optimization |
| | mobaraense | |
| 2005 | Streptoverticillium | Environmental control strategies |
| | mobaraense | |
| 2006 | Bacillus circulans | Strain isolation and substrate |
| | | optimization |
| 2007 | Streptomyces sp. | Strain isolation and substrate |
| | | optimization |
| 2007 | Streptomyces | Strain isolation and environmental |
| | hygroscopicus | control strategies |
| 2008 | Several Streptomyces | Solid fermentation |
| 2009 | Streptomyces | Fermentation strategies |
| | hygroscopicus | |
| 2017 | Kutzneria albida | Substrate optimization |

A Generally Recognized as Safe (GRAS) status has been assigned to Tgase preparations from S. mobaraensis for protein crosslinking in seafood, meat, dairy, and cereal products (FDA/CFSAN agency response letters: GRAS notice numbers 000004 (1998), 000029 (1999), 000055 (2001), and 000095 (2002)). Commercially available microbial transglutaminase is produced on large scale and distributed under the trade name ACTIVA® by Ajinomoto US, Inc.

Lysyl oxidases (LOX, EC 1.4.3.13, also known as protein-lysine 6-oxidase) are copper-dependent enzymes that oxidize primary amine substrates to reactive aldehydes. Five different LOX enzymes have been identified in mammals, LOX and LOX-like (LOXL) 1 to 4, showing a highly conserved catalytic carboxy terminal domain and more divergence in the rest of the sequence. Additionally, LOX proteins have been identified in many other eukaryotes, as well as in bacteria and archaea, reviewed in Grau-Bove, et al. (2015) Scientific Reports 5: Article number: 10568.

Tyrosinase (EC 1.14.18.1) is a copper-containing oxidase that oxidizes phenols, such as tyrosine and dopamine, to form reactive o-quinones, which readily form crosslinks with solvent-exposed lysyl, tyrosyl, and cysteinyl residues, as well as numerous small molecules. In nature, tyrosinase plays a crucial role in sclerotization and melanization, and is perhaps best known as the enzyme responsible for the enzymatic browning of fruits and vegetables. Tyrosinase has been demonstrated to induce crosslinking of the whey proteins α-lactalbumin and β-lactoglobulin. Tyrosinases have been isolated and studied from a wide variety of plant, animal, and fungal species.

The best known and characterized tyrosinases are of mammalian origin. The most extensively investigated fungal tyrosinases, both from a structural and functional point of view, are from Agaricus bisporus (Wichers, et al. (1996) Phytochemistry 43(2):333-337) and Neurospora crassa (Lerch (1983) Mol Cell Biochem 52(2):125-128). A few bacterial tyrosinases have been reported, of which Streptomyces tyrosinases are the most thoroughly characterized (U.S. Pat. Nos. 5,801,047 and 5,814,495). In addition, tyrosinases have been disclosed, e.g., from Bacillus and Myrothecium (EP919628), Mucor (JP61115488), Miriococcum (JP60062980), Aspergillus, Chaetotomastia, and Ascovaginospora (Abdel-Raheem and Shearer (2002) Fungal Diversity 11(5):1-19), and Trametes (Tomsovsky and Homolka (2004) World Journal of Microbiology and Biotechnology 20(5):529-530).

Laccases are multi-copper oxidases found in plants, fungi, and bacteria, which oxidize phenolic substrates, performing one-electron oxidations, resulting in crosslinking. Methods for crosslinking proteins by laccases have been disclosed, e.g., in US2002/009770. Plant proteins derived from beans, cereals, and animal proteins, including milk, egg, meat, blood, and tendon are listed as suitable substrates. Fungal laccases are disclosed in US2002/019038.

Sortases constitute a group of calcium-dependent enzymes embedded in the membrane of Gram-positive bacteria. Based on their primary amino acid sequences, sortases are currently assigned to six different classes (A-F) that exert highly site-specific transpeptidation reactions at the bacterial cell surface (Spirig, et al. (2011) Mol Microbiol 82:1044-1059). These include the anchoring of diverse functional proteins to the growing cell wall by sortase A (Marraffini, et al. (2006) Microbiol Mol Rev 70:192-221; Mazmanian, et al. (1999) Science 285:760-763) and the assembly of pili from individual pilin subunits by sortase C (Hendrickx, et al. (2011) Nat Rev Microbiol 9:166-176).

Formylglycine-Generating Enzyme (FGE, EC 1.8.3.7) is a copper-containing oxidase that catalyzes the cotranslational or posttranslational activation of type I sulfatases in eukaryotes and aerobic microbes (Appel et al. (2019) Proc Natl Acad Sci 116(12): 5370-5375). This is accomplished by oxidation of a sulfatase active-site cysteine residue to formylglycine (fGly). The promiscuity of FGE has enabled its use in biotechnology and therapeutic applications, such as site-specific drug attachment to fGly in monoclonal antibodies.

Sulfhydryl Oxidase (SOX, EC 1.8.3.2) oxidizes the free sulfhydryl groups in proteins and thiol-containing small molecules by using molecular oxygen as an electron acceptor (Faccio et al. (2011) App Microbiol Biotechnol 91(4) 957-966). SOXs have been isolated from the intracellular compartments of many organisms, where they form disulfide bridges between proteins. Additionally, SOXs have been found in the secretomes of many industrially relevant organisms.

As used herein the term “percent identity” is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the number of matching nucleotides or amino acids between strings of such sequences. “Identity” and “similarity” can be readily calculated by known methods, including but not limited to those described in: Computational Molecular Biology (Lesk, A M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991).

Methods to determine identity and similarity are codified in publicly available computer programs.

As used herein, “% identity” or “percent identity” or “PID” refers to protein sequence identity. Percent identity may be determined using standard techniques known in the art. Useful algorithms include the BLAST algorithms (See Altschul, et al., J Mol Biol, 215:403-410, 1990; and Karlin and Altschul, Proc Natl Acad Sci USA, 90:5873-5787, 1993). The BLAST program uses several search parameters, most of which are set to the default values. The NCBI BLAST algorithm finds the most relevant sequences in terms of biological similarity but is not recommended for query sequences of less than 20 residues (Altschul, et al., Nucleic Acids Res, 25:3389-3402, 1997; and Schaffer et al., Nucleic Acids Res, 29:2994-3005, 2001). Exemplary default BLAST parameters for a nucleic acid sequence searches include: Neighboring words threshold=11; E-value cutoff=10; Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. A percent (%) amino acid sequence identity value is determined by the number of matching identical residues divided by the total number of residues of the “reference” sequence. BLAST algorithms refer to the “reference” sequence as the “query” sequence.

As used herein, “homologous proteins” refers to proteins that have distinct similarity in primary, secondary, and/or tertiary structure. Protein homology can refer to the similarity in linear amino acid sequence when proteins are aligned. Homologous search of protein sequences can be done using BLASTP and PSI-BLAST from NCBI BLAST with threshold (E-value cut-off) at 0.001. Gapped BLAST and PSI BLAST are a new generation of protein database search programs (Altschul S F, Madde T L, Shaffer A A, Zhang J, Zhang Z, Miller W, Lipman D J, Nucleic Acids Res (1997) 25(17):3389-402). Using this information, protein sequences can be grouped.

Sequence alignments and percent identity calculations may be performed using the Megalign program of the LASERGENE bioinformatics computing suite (DNASTAR Inc., Madison, WI), the AlignX program of VectorNTI v. 7.0 (Informax, Inc., Bethesda, MD), or the EMBOSS Open Software Suite (EMBL-EBI; Rice et al., Trends in Genetics 16, (6):276-277 (2000)). Multiple alignment of the sequences can be performed using the CLUSTAL method (such as CLUSTALW; for example, version 1.83) of alignment (Higgins and Sharp, CABIOS, 5:151-153 (1989); Higgins et al., Nucleic Acids Res. 22:4673-4680 (1994); and Chenna et al., Nucleic Acids Res 31 (13):3497-500 (2003)), available from the European Molecular Biology Laboratory via the European Bioinformatics Institute) with the default parameters. Suitable parameters for CLUSTALW protein alignments include GAP Existence penalty=15, GAP extension=0.2, matrix=Gonnet (e.g., Gonnet250), protein ENDGAP=−1, protein GAPDIST=4, and KTUPLE=1. In one embodiment, a fast or slow alignment is used with the default settings where a slow alignment. Alternatively, the parameters using the CLUSTALW method (e.g., version 1.83) may be modified to also use KTUPLE=1, GAP PENALTY=10, GAP extension=1, matrix=BLOSUM (e.g., BLOSUM64), WINDOW=S, and TOP DIAGONALS SAVED=5.

Alternatively, multiple sequence alignment may be derived using MAFFT alignment from Geneious® version 10.2.4 with default settings, scoring matrix BLOSUM62, gap open penalty 1.53 and offset value 0.123.

The MUSCLE program (Robert C. Edgar. MUSCLE: multiple sequence alignment with high accuracy and high throughput (Nucl. Acids Res. (2004) 32(5):1792-1797) is yet another example of a multiple sequence alignment algorithm.

Examples of components optionally having antimicrobial activity include, but are not limited to, proteases, hydrolyases and lyases, such as lysozymes, chitinases, lipases, lysins, lysostaphins, subtilisins, amylases, cellulases, glucanases, DNases, RNases, lactoferrins, glucose oxidases, peroxidases, lactoperoxidases, lactonases, acylases, dispersin B, amylases, cellulases, nisins, bacteriocins, siderophores, polymyxins, and defensins.

Examples of chemical preservatives that are suitable for use in the compositions described herein include, but are not limited to, quaternary ammonium compounds, detergents, chaotropic agents, organic acids, alcohols, glycols, aldehydes, oxidizers, parabens, isothiazolinones, and cationic polymers.

In another embodiment, the crosslinking enzyme is in the form of a zymogen and the composition comprises an enzyme, wherein the zymogen and the enzyme interact to produce an active or mature enzyme (e.g., the zymogen and the enzyme interact such that the zymogen is converted to a mature form of the zymogen) having preservative and/or antimicrobial activity.

In still another embodiment, there is disclosed an expression vector comprising at least one heterologous nucleic acid sequence that encodes at least one crosslinking enzyme, optionally in the form of a zymogen, wherein said heterologous nucleic acid sequence is optionally operably linked to at least one regulatory sequence wherein the expression vector is capable of transforming a host cell to express, either intracellularly or extracellularly, said at least one crosslinking enzyme so that the transformed host cell is inactivated, inhibited (e.g., growth of the transformed host cell is inhibited), or killed.

Preservatives are antimicrobial ingredients added to product formulations to maintain the microbiological safety of the products by inhibiting the growth of and reducing the amount of microbial contaminants. US Pharmacopeia has published protocols for acceptable microbial survival for preservatives in cosmetics and personal care products. These tests include USP 51 (Antimicrobial Effectiveness Test) and USP 61 (Microbial Limits Test) (https://www.fda.gov/files/about %20fda/published/Pharmaceutical-Microbiology-Manual.pdf).

The effectiveness of the preservative system disclosed herein is determined based on the MIC (minimum inhibitory concentration) against a variety of microbes, including, but not limited to, Gram-positive bacteria, Gram-negative bacteria, yeast and/or mold (e.g., S. aureus ATCC 6538, E. coli ATCC 8739, P. aeruginosa ATCC 9027, C. albicans ATCC 10231, and A. brasiliensis ATCC 16404). Minimum inhibitory concentrations (MICs) are defined as the lowest concentration of an antimicrobial that will inhibit the growth of a microorganism. Microbial growth may be determined, for example, by spectrophotometric methods (the optical density at 600-650 nm) or with a cell viability assay (e.g., BacTiter-Glo™, Promega®).

In some embodiments, the at least one crosslinking enzyme utilized in a composition described herein is initially in the form of a zymogen. As discussed above, zymogens are inactive enzyme precursors (proenzymes) that are expressed with a pro-sequence that must be cleaved to afford active enzyme having the desired antimicrobial and/or preservative activity. Cleavage of a pro-sequence affords an active or mature enzyme (i.e., a mature form of the zymogen) that is often highly toxic to cells. A proenzyme is expressed with a cleavable leader sequence to suppress activity of the enzyme, due to related enzyme toxicity to the cell. Therefore, zymogens present a useful class of enzymes for use as antimicrobial or preservative agents if their mature form exhibits antimicrobial properties. The mature active enzyme form (i.e., without the pro-sequence) may be used in the disclosed compositions for preparation of an antimicrobial and/or preservative composition. Useful enzymes within this category include, but are not limited to, lytic enzymes (e.g. proteases, hydrolases, lyases, nucleases) and crosslinking enzymes.

In some embodiments, an inactive zymogen (e.g., a crosslinking enzyme, such as a zymogen of a transglutaminase, laccase, peroxidase, transferase, lysyl oxidase, tyrosinase, sortase, formylglycine-generating enzyme, or sulfhydryl oxidase), as described herein, is combined with at least one enzyme, such as a protease, in a composition or product or in a preservative or antimicrobial method of use. The zymogen may be stored with the enzyme together in a composition or may be combined at the site of use. The enzyme may serve to activate the zymogen (e.g., interact with the zymogen to convert the zymogen to a mature form of the zymogen), for example, for preservation of a product or in an antimicrobial application of use (e.g., for microbial control).

In some embodiments, the compositions include one or more antimicrobial agents, such as an enzyme, a peptide and the like. An example of an antimicrobial agent includes, but is not limited to, chitosan. Without being bound by theory, the use of an antimicrobial agent may have an additive effect together with antimicrobial activity of the crosslinking enzyme or enzymes present, delivering broad spectrum microbial control. Chitosan, for example, ruptures the cell membrane and leads to spillage of the cell contents. A crosslinking enzyme, as described herein, can crosslink proteins vital for cell function both on the surface of the cell and within the cell. This combination of both materials together (i.e., an antimicrobial enzyme and an antimicrobial agent (e.g., chitosan)) reduces the quantity of the materials needed (i.e., less antimicrobial chemical (e.g., chitosan) and less crosslinking enzyme) and provides additional stability to the enzyme, allowing for greater activity over time, and reduces the undesirable effects that may accompany the use of an antimicrobial chemical, such as chitosan.

Nonlimiting examples of known antimicrobial enzymes, peptides, and proteins, which may be included in the compositions described herein, are shown in Table 3.

TABLE 3
Enzymes, Peptides and Proteins with Known Antimicrobial Properties

| Mechanism | Enzyme | Description | Citation |
|---|---|---|---|
| Lytic | Lysozyme | Produced by animals as | Ibrahim et al. (2001) FEBS |
| | | part of the innate immune | Letters 506(1): 27-32; |
| | | system. Hydrolyzes the | Małaczewska et al. (2019) |
| | | peptidoglycan subunits in | BMC Vet. Res. 15: 318 |
| | | the bacterial cell wall. | |
| | Chitinase | Secreted by soil bacteria | Martínez-Zavala et al (2020) |
| | | including Bacillus | Front. Microbiol. 10: 3032 |
| | | thuringiensis to combat | |
| | | insects and fungi | |
| | Lipase | Hydrolyzes extracellular | Prabhawathi et al. (2014) |
| | | lipids and polymers. | PLoS One 9(5) |
| | Lysin | Utilized by bacteriophages | Hoops et al. (2008) Appl. |
| | | to hydrolyze the glycan | Environ. Microbiol. 75: 5, |
| | | component of bacterial cell | 1388-1394 |
| | | wall | |
| | Lysostaphin | Metalloendopeptidase | Kokai-Kun et al. (2003) |
| | | which cleaves the | Antimicrob Agents |
| | | pentaglycine bridges found | Chemother 47(5): 1589-1597 |
| | | in cell wall peptidoglycan. | |
| | Glucanase | Secreted by soil bacteria | Shafi et al. (2017) |
| | | including Bacillus species | Biotechnology & |
| | | to degrade the fungal cell | Biotechnological Equipment |
| | | wall. Has also been utilized | 31: 3 446-459 |
| | | as an algicide and for | |
| | | biofilm control. | |
| Nuclease | DNase | Hydrolyzes extracellular | Kaplan et al. (2012) J. |
| | | nucleic acids and viral | Antibiot. (Tokyo) 65(2): 73- |
| | | genomic DNA. | 77 |
| | RNase | Hydrolyzes viral RNA. | Wirth (1992) |
| | | | WO1994000016A1 |
| | Lactoferrin | Sequesters essential iron | Niaz et al. (2019) |
| | | ions to prevent microbial | International Journal of |
| | | growth. Also possesses | Food Properties 22: 1 1626- |
| | | nuclease activity and | 1641 |
| | | hydrolyzes biofilm | |
| | | polymers. | |
| Oxidoreductase | Glucose Oxidase | Oxidizes glucose to D- | Wong et al. (2008) Appl |
| | | glucono-δ-lactone and | Microbiol Biotechnol. |
| | | hydrogen peroxide. | 78(6): 927-938 |
| | Peroxidase | Oxidizes inert substrates to | Ihalin et al. (2006) Arch. |
| | | form biocidal actives. | Biochem. Biophys. 445, 261- |
| | | | 268 |
| | Lactoperoxidase | Oxidizes inert substrates to | White et al. (1983) |
| | | form biocidal actives. | Antimicrob Agents |
| | | | Chemother 32(2): 267-272 |
| Quorum | Lactonase | Hydrolyzes quorum | Schwab et al. (2019) Front |
| Quenching | | sensing lactones, | Microbiol. 10: 611 |
| | | preventing activation of | |
| | | biofilm- and pathogenesis- | |
| | | promoting pathways. | |
| | Acylase | Hydrolyzes quorum | Vogel et al. (2020) Front. |
| | | sensing lactones, | Chem. 8: 54 |
| | | preventing activation of | |
| | | biofilm- and pathogenesis- | |
| | | promoting pathways. | |
| Hydrolase | Dispersin B | Hydrolyzes biofilm | Izano et al. (2007) J Dent |
| | | polymers | Res 86(7): 618-622 |
| | α-amylase | Hydrolyzes extracellular | Craigen et al. (2011) Open |
| | | polysaccharides. | Microbiol J. 5: 21-31 |
| | Cellulase | Hydrolyzes the cellulose | Loiselle et al. (2003) |
| | | component of biofilms and | Biofouling 19(2): 77-85 |
| | | algal cell walls. | |
| Antimicrobial | Nisin | Increases permeability of | Li et al. (2018) Appl Environ |
| Peptides | | the microbial cell | Microbiol 18(12) |
| | | membrane. | |
| | Bacteriocin | Modes of action include | Meade et al. (2020) |
| | | inhibition of cell wall | Antibiotics 9(1): 32 |
| | | synthesis and increasing | |
| | | cell membrane | |
| | | permeability. | |
| | Siderophore | Binds to and sequesters | Raaska et al. (1999) J Indust |
| | | iron ions | Microbiol Biotechnol 22, |
| | | | 27-32 |
| | Polymyxin | Increases permeability of | Poirel et al. (2017) Clin |
| | | the microbial cell | Microbiol Rev 30: 577-596 |
| | | membrane. | |
| | Defensin | Increases permeability of | Gans (2003) Nat Rev |
| | | the microbial cell | Immunol 3, 710-720 |
| | | membrane. | |

In some embodiments, any of the crosslinking enzymes disclosed herein, such as, but not limited to, a transglutaminase, a lysyl oxidase, a tyrosinase, a laccase, a sortase, a formylglycine-generating enzyme, or a sulfhydryl oxidase, may be utilized in an antimicrobial and/or preservative composition in combination with one or more of the antimicrobial enzymes, peptides, or proteins described in Table 3 to provide broad spectrum microbial control.

The compositions described herein may include antimicrobial chemicals. An antimicrobial crosslinking enzyme, as described herein, such as, but not limited to, a transglutaminase, a lysyl oxidase, a tyrosinase, a laccase, a sortase, a formylglycine-generating enzyme, or a sulfhydryl oxidase, may be formulated with one or more antimicrobial chemical(s), including, but not limited to chitosan, polylysine, or quaternary ammonium compounds, for example, for use as an antimicrobial composition. Nonlimiting examples of antimicrobial chemicals are shown in Table 4 below.

TABLE 4
Examples of Antimicrobial Chemicals
for Antimicrobial Applications

| Classification | Chemical |
|---|---|
| Polymers | Chitosan |
| | N,N,N-trimethyl chitosan |
| | ε-poly-lysine |
| | Polyvinylbenzyl-dimethylbutyl ammonium chloride |
| | Polyvinylbenzyl trimethyl ammonium chloride |
| | Quaternary ammonium polyethyleneimine |
| | Quaternary phosphonium modified epoxidized |
| | natural rubber |
| | Arginine-tryptophan-rich peptide |
| | Guanylated polymethacrylate |
| | Ammonium ethyl methacrylate homopolymers |
| | Metallo-terpyridine carboxymethyl cellulose |
| | Poly(n-vinylimidazole) modified silicone rubber |
| Quaternary | Cocoamidopropyl Betaine |
| Ammonium | Myristamidopropyl-pg-dimonium Cl Phosphate |
| | Benzalkonium Chloride (BZK) |
| | Quaternium-6 |
| | Coco Betaine |
| Detergents | Sodium Lauryl Sulfate |
| | Dodecylbenzenesulfonic Acid |
| Chaotropic Agent | Polyamidopropyl biguanide |
| | Guanidinium chloride |
| Organic Acids | Lactic Acid |
| | Citric Acid |
| | Salicylic Acid |
| | Sorbic Acid |
| | Acetic Acid |
| | Dehydroacetic Acid |
| | Peracetic Acid |
| | Benzoic Acid |
| Phenols & Alcohols | Ethanol |
| | Isopropanol |
| | Dichlorobenzyl Alcohol |
| | Glycerol |
| | Caprylyl Glycol |
| | Ethylhexylglycerin |
| | Benzyl Alcohol |
| | 2-Phenoxyethanol |
| Aldehydes & | Glutaraldehyde |
| Aldehyde | Formaldehyde |
| Releasers | Sodium Hydroxymethylglycerate |
| | DMDM Hydantoin |
| Base | Sodium Hydroxide |
| Oxidizers | Hydrogen Peroxide |
| Parabens | Methyl Paraben |
| | Ethyl Paraben |
| | Propyl Paraben |
| Misc | Natamycin |
| | Benzisothiazolinone |
| | Bronopol |
| | Sorbitan Caprylate |
| | Ethyl Lauroyl Arginate |
| | Methylisothiazolinone (MIT) |
| | Cetylpyridinium Chloride |
| | Chlorphenesin |
| | Zinc Omadine |
| | Sodium Omadine |
| | N-(3-aminopropyl)-N-dodecylpropane-1,3-diamine |
| | Methylchloroisothiazolinone |
| | 2,2-dibromo-3-nitrilopropionamide |
| | 1-Octadecanaminium, N,N-dimethyl-N-[3- |
| | (trimethoxysilyl)propyl]-, chloride |
| | Saponin |
| | Sodium Benzoate |

Cationic biopolymers and quaternary ammonium compounds have been successfully employed as preservatives or preservative potentiators owing to their ability to disrupt cell membranes. Natural cationic biopolymers, like chitosan, are well known for their antimicrobial activity (Kong, et al. (2010) Int. J. of Food Microbiol. 144: 51-63). The antimicrobial activity of chitosan against different groups of microorganisms, such as bacteria, yeast, and fungi, is known. Quaternary ammonium compounds (non-limiting examples include, cetyl pyridinium chloride, benzethonium chloride, benzalkonium chloride, and polyaminopropyl biguanide), similarly have notable antimicrobial properties. These quaternary ammonium compounds, however, have limited use for the personal care industry due to specific incompatibilities with other cosmetic ingredients. For example, benzethonium chloride is deactivated by many anionic ingredients, such as anionic surfactants, that form important part of topical personal care formulations.

Crosslinkers such as formaldehyde and formaldehyde donors like DMDM hydantoin (CAS 6440-58-0), imidazolidinyl urea, and diazolidinyl urea (CAS 39236-46-9) are also used. The formaldehyde released by these substances is capable of reacting with several cosmetic ingredients via its reactive aldehydic carbonyl functionality, in addition to health concerns limiting the wide spread use of formaldehyde. For example, Avobenzone reacts with formaldehyde that is released by formaldehyde derivatives.

Parabens are esters of p-hydroxybenzoic acid. Paraben compounds include Methyl-paraben (CAS 99-76-3), Ethyl-paraben (CAS 120-47-8), Propyl-paraben (CAS 94-13-3), Butyl-paraben (CAS 94-26-8), Isopropyl-paraben (CAS 4191-73-5), and Benzyl-paraben (CAS 94-18-8). Parabens are phenol derivatives, which have a phenolic ‘hydroxyl’ group with pKₐ of 10 that can react with organic functionality. Additionally, parabens have lost consumer favor owing to their possible role as endocrine disruptors.

Halogenated molecules, such as chlorothiazolinones, 2,4-dichlorobenzyl-alcohol, chloroxylenol, methyl dibromo glutaronitrile, 2-bromo-2-nitro-1,3-diol, chlorphenesin, and chlorhexidine, are highly reactive compounds and their usage levels have been highly regulated across the personal care industry to limit toxicity and sensitization. For example, IPBC has risk of thyroid hormonal disturbances due to its iodine content. It has not been allowed in Japan and in the EU is allowed only up to 0.02% in leave-on products. Similarly, the EU permits usage of methyl dibromo glutaronitrile only up to 0.1% in rinse-off products only. Bronopol, 2-bromo-2-nitropropane-1,3-diol, is implicated in generation of carcinogenic nitrosoamines on interacting with some of the nitrogen containing cosmetic ingredients. The antimicrobial efficacy of methylchloroisothiazolinone is allowed only in rinse-off products at 15 ppm concentration.

It should be noted that the broad spectrum antimicrobial and/or preservative compositions disclosed herein may be used in a variety of applications such as personal care, household, industrial, institutional, oil and gas, marine, food and beverage, agricultural, animal, and human nutrition, water purification and the like.

Non-limiting embodiments of the foregoing disclosed herein include:
1. A composition comprising (a) at least one crosslinking enzyme, optionally in the form of a zymogen, in combination with (b) at least one component selected from the group consisting of enzymes, peptides, and/or proteins, optionally having antimicrobial activity, and optionally further in combination with (c) at least one chemical preservative, wherein the composition comprising (a) in combination with (b) and optionally, further in combination with (c), has at least one activity selected from the group consisting of preservative and antimicrobial.
2. The composition of embodiment 1 wherein the at least one crosslinking enzyme is selected from the group consisting of transglutaminases, lysyl oxidases, tyrosinases, laccases, sortases, formylglycine-generating enzymes, and sulfhydryl oxidases.
3. The composition of embodiment 1 or 2 wherein the at least one crosslinking enzyme is a transglutaminase.
4. The composition of embodiment 1, 2 or 3 wherein the transglutaminase has at least 90% sequence identity with the amino acid sequence in SEQ ID NO:2.
5. The composition of embodiment 1, 2, 3 or 4 wherein the at least one component having antimicrobial activity is selected from the group consisting of lysozymes, chitinases, lipases, lysins, lysostaphins, glucanases, DNases, RNases, lactoferrins, glucose oxidases, peroxidases, lactoperoxidases, lactonases, acylases, dispersin B, amylases, proteases, cellulases, nisins, bacteriocins, siderophores, polymyxins, and defensins.
6. The composition of embodiment 1, 2, 3, 4 or 5 wherein the at least one chemical preservative is selected from the group consisting of quaternary ammonium compounds, detergents, chaotropic agents, organic acids, alcohols, glycols, aldehydes, oxidizers, and parabens.
7. The composition of embodiment 1, 2, 3, 4, 5 or 6 wherein (a) is a zymogen and (b) comprises an enzyme further wherein the zymogen and the enzyme interact to produce an active enzyme of the zymogen having at least one activity selected from the group consisting of preservative and antimicrobial.
8. An expression vector comprising at least one heterologous nucleic acid sequence that encodes at least one crosslinking enzyme, optionally in the form of a zymogen, wherein said heterologous nucleic acid sequence is optionally operably linked to at least one regulatory sequence and wherein the expression vector is capable of transforming a host cell to express, either intracellularly or extracellularly, said at least one crosslinking enzyme so that the transformed host cell is inactivated, inhibited, or killed.
9. The expression vector of embodiment 8 wherein the at least one crosslinking enzyme is selected from the group consisting of transglutaminases, lysyl oxidases, tyrosinases, laccases, sortases, formylglycine-generating enzymes, and sulfhydryl oxidases.
10. The expression vector of embodiment 8 or 9 wherein the at least one crosslinking enzyme is a transglutaminase.
11. The expression vector of embodiment 8, 9 or 10 wherein the transglutaminase has at least 90% sequence identity with the amino acid sequence in SEQ ID NO:2.
12. The expression vector of embodiment 8, 9, 10 or 11 wherein the at least one component having antimicrobial activity is selected from the group consisting of lysozymes, chitinases, lipases, lysins, lysostaphins, glucanases, DNases, RNases, lactoferrins, glucose oxidases, peroxidases, lactoperoxidases, lactonases, acylases, dispersin B, amylases, proteases, cellulases, nisins, bacteriocins, siderophores, polymyxins, and defensins.
13. The expression vector of embodiment 8, 9, 10, 11 or 12 wherein the at least one chemical preservative is selected from the group consisting of quaternary ammonium compounds, detergents, chaotropic agents, organic acids, alcohols, glycols, aldehydes, oxidizers, parabens, isothiazolinones, and cationic polymers.
14. The expression vector of embodiment 8, 9, 10, 11, 12, or 13 wherein (a) is a zymogen and (b) comprises an enzyme further wherein the zymogen and the enzyme interact to produce an active enzyme having at least one activity selected from the group consisting of preservative and antimicrobial.
15. The expression vector of embodiment 8, 9, 10, 11, 12, 13, or 14 wherein the transglutaminase has at least 90% sequence identity with the amino acid sequence in SEQ ID NO:2.

The following examples are intended to illustrate, but not limit, the invention.

### EXAMPLES

Unless defined otherwise herein, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used with this disclosure.

The disclosure is further defined in the following Examples. It should be understood that these Examples, while indicating certain embodiments, are given by way of illustration only.

From the above discussion and the Examples, one skilled in the art can ascertain essential characteristics of this disclosure, and without departing from the spirit and scope thereof, can make various changes and modifications to adapt to various uses and conditions.

### Examples

### Example 1. Antimicrobial Properties of Wild-Type Tgase (SEQ ID NO:1)

Commercially available wild-type Streptomyces mobaraensis transglutaminase (TI formulation) having the amino acid sequence depicted SEQ ID NO:1 was sourced from Ajinomoto. Tgase is available from Ajinomoto USA under the trade name Activa®-TI. This product is sold as a solid preparation of 99% maltodextrin and 1% microbial enzyme. Ajinomoto reports the enzyme activity is 81-135 U/g. The Activa®-TI was used as received as well as purified from the maltodextrin by tangential flow filtration or diafiltration to concentrate the enzyme. Additionally, wild-type Tgase SEQ ID NO:1 has been prepared by literature methods (Javitt, et al. (2017) BMC Biotechnol. 17:23) as previously described. Tgase activity was measured using the colorimetric hydroxamate activity assay (Folk and Cole (1965) J Biol Chemistry 240(7):2951-2960). Both preparations provided similar results.

E. coli ATCC 8739 and C. albicans ATCC 10231 were acquired from the American Type Culture Collection (ATCC) (Manassas, VA) and maintained as −80° C. frozen glycerol stocks. B. subtilis BGSC 1A1276 was purchased from the Bacillus Genetic Stock Center (BGSC) (Columbus, OH) and maintained as −80° C. frozen glycerol stock. E. coli DH5-alpha and E. coli DH10-beta were purchased from New England Biolabs (NEB) (Ipswich, MA) and maintained as −80° C. frozen glycerol stock.

For MIC determination of bacterial cultures, E. coli ATCC 8739, DH5-alpha, DH10-beta, and B. subtilis BGSC 1A1276 was grown overnight (16-18 hours) in LB broth at 37° C. The following day, the cell density of the saturated cultures was calculated using OD₆₀₀ and cultures were diluted to 10⁴ to 10⁶ CFU/mL in sterile LB media to generate the inoculum, and 90 μL of the inoculum was combined with 10 μL of serially diluted Tgase SEQ ID NO:1 at a range of 0.0001-0.01 weight percent in the presence or absence of 0.003% lysozyme (100-fold lower than the effective concentration). Growth curves were measured by OD₆₀₀ on a BioTek® Synergy Plate Reader. Optionally, the following day, a cell viability assay such as BacTiter-Glo™ (Promega®) following manufacturer's protocols) could be used to assess cell viability. A decrease in OD₆₀₀ or luminescence indicated a decrease in cell viability. Results are presented as a percent reduction in cell count relative to an untreated culture. All test conditions were performed in triplicate.

For MIC determination of yeast cultures, C. albicans ATCC 10231 was grown overnight (24 hours) in YPD media at 30° C. The following day, the cell density of the saturated cultures was calculated using OD₆₀₀ and cultures were diluted to 10⁴ to 10⁶ CFU/mL in sterile YPD media to generate the inoculum, and 90 μL of the inoculum was combined with 10 μL of serially diluted Tgase SEQ ID NO:1 at a range of 0.0001-0.01 weight percent. The cultures were grown overnight at 30° C. and growth curves were measured by OD₆₀₀ on a BioTek® Synergy Plate Reader. Optionally, the following day, a cell viability assay such as BacTiter-Glo™ (Promega®) following manufacturer's protocols) could be used to assess cell viability. A decrease in OD₆₀₀ or luminescence indicated a decrease in cell viability. Results are presented as a percent reduction in cell count relative to an untreated culture. All test conditions were performed in triplicate.

When gram negative E. coli (ATCC 8739, DH5-alpha, and DH10-beta) were grown in the presence or absence of Tgase SEQ ID NO:1, Tgase SEQ ID NO:1 showed little or no detectable antimicrobial activity under the concentrations evaluated. Cloning strains, DH5-alpha and DH10-beta, were utilized to represent cells with more accessible cell membranes, as these strains have vulnerable cell matrices and are more conducive to penetration of the cell membrane by macromolecules such as DNA.

When using the gram-positive bacterial cloning strain, B. subtilis BGSC 1A1276, antimicrobial activity of Tgase SEQ ID NO:1 was observed. Full inhibition of B. subtilis BGSC 1A1276 growth was observed upon adding a cell membrane disruptor, lysozyme. Growth of both C. albicans and B. subtilis is partially inhibited by Tgase SEQ ID NO:1. Results are listed in Table 5.

### Example 2. Antimicrobial Properties of Tgase Variant (SEQ ID NO:6)

A variant form of Streptomyces mobaraensis transglutaminase having the amino acid sequence depicted in SEQ ID NO:6 was prepared by literature methods (Javitt, et al. (2017) BMC Biotechnol. 17:23) as previously described. Tgase activity was measured using the colorimetric hydroxamate activity assay (Folk and Cole (1965) J Biol Chemistry 240(7):2951-2960). Tgase cytotoxic activity was assessed either using OD₆₀₀ or a commercially available kit (BacTiter-Glo™, Promega®, following manufacturer's protocol).

Yeast or bacterial starter cultures were grown as described previously. Tgase (SEQ ID NO:6) was added to each culture at 0.001-1 weight percent. The cultures were grown overnight at 30° C.-37° C. and growth curves were measured by a BioTek® Synergy Plate Reader. Tgase SEQ ID NO:6 was found to be 10-fold more potent than Tgase SEQ ID NO:1 where biocidal activity could be observed with both enzymes. See table 5.

TABLE 5
Antimicrobial efficacy of wild-type Tgase in combination with lysozyme

| | | Enzyme | Percent | |
|---|---|---|---|---|
| | | Concentration | Growth | Fold improvement |
| Condition | Strain | (ug/mL) | Reduction | in enzyme efficacy |
| Tgase (SEQ ID | B. subtilis | 960 | 99.17 | 1 |
| NO: 1) | BGSC 1A1276 | | | |
| Tgase (SEQ ID | C. albicans | 800 | 83.11 | 1 |
| NO: 1) | ATCC 10231 | | | |
| Tgase (SEQ ID | B. subtilis | 960 Tgase | 99.81 | 4.4 |
| NO: 1) with | BGSC 1A1276 | 300 Lysozyme | | |
| Lysozyme | | | | |
| Tgase (SEQ ID | B. subtilis | 80 | 99.77 | 12 |
| NO: 6) | BGSC 1A1276 | | | |
| Tgase (SEQ ID | C. albicans | 80 | 99.95 | 10 |
| NO: 6) | ATCC 10231 | | | |

### Example 3. SEQ ID NO:6 Minimum Inhibitory/Fungicide Concentration

A variant form of Streptomyces mobaraensis transglutaminase having the amino acid sequence depicted in SEQ ID NO:6 was prepared by literature methods with a hexa-his-tag to aid in purification (Javitt, et al. (2017) BMC Biotechnol. 17:23). The cells were grown in shake flasks, lysed by homogenization, and the Tgase variant (SEQ ID NO:6) was isolated from the cell debris by centrifugation. The resulting semi-purified enzyme (clarified lysate) were compared on an SDS-PAGE gel, by spectroscopy, and activity for concentration of active enzyme. The Tgase variant (SEQ ID NO:6) was further purified by affinity column on a Ni-IMAC resin prior to MIC assay. Tgase activity was measured in the examples herein using a colorimetric hydroxamate activity assay (Folk and Cole (1965) J Biol Chemistry 240(7):2951-2960). The enzyme was diluted to a working stock concentration of 2 mg/mL in CAMHB or RPMI media (bacterial or fungal media). This was then 2-fold serial diluted ten times in the appropriate media in a 96-well master plate to generate 2× starting concentrations for MIC testing.

Benzalkonium chloride (BZK) was sourced from Sigma-Aldrich® and sodium benzoate was sourced from Emerald Kalama Chemical under the tradename Kalaguard SB.

Strains were acquired from the American Type Culture Collection (ATCC) (Manassas, VA) and maintained as −80° C. frozen glycerol stocks: S. aureus ATCC 6538, E. coli ATCC 8739, P. aeruginosa ATCC 9027, C. albicans ATCC 10231, and A. brasiliensis ATCC 16404. B. subtilis BGSC 1A1276 was purchased from the Bacillus Genetic Stock Center (BGSC) (Columbus, OH) and maintained as −80° C. frozen glycerol stock. Cation-adjusted Mueller-Hinton broth (CAMHB) at pH 7.3 was used for testing with bacterial species (unless otherwise noted), while RPMI 1640 media buffered with 0.165 M MOPS at pH 7.0 was used for testing with all fungal species.

For MIC determination, bacterial strains (S. aureus ATCC 6538, E. coli ATCC 8739, P. aeruginosa ATCC 9027) were grown overnight on Tryptic Soy Agar (TSA) plates at 37° C. A few colonies of each strain were collected with a sterile swab and used to make a McFarland 0.5 standard solution (approximately 1×10⁸ CFU/mL) in sterile PBS. The McFarland solution was then diluted 1:100 in CAMHB to generate the inoculum, and 50 μL of this inoculum was combined with 50 μL of the 2× test article in a 96-well plate for a final concentration of 1×. All test conditions were performed in duplicate. The bacterial-strain MIC 96-well plates were incubated at 37° C. for 18 to 20 hours. MIC is defined as the lowest concentration of a compound in which no visible growth is observed. Results are presented in Tables 6 and 7.

For MIC determination of fungal strains, A. brasiliensis and C. albicans were grown on Sabouraud Dextrose Agar (SDA) plates. A. brasiliensis was grown at 25° C. for 5 to 7 days, while C. albicans was grown at 37° C. for 24 to 48 hours.

A. brasiliensis was harvested from the SDA plate with a sterile swab into PBS after 7 days of growth. This suspension was then allowed to stand for 5 to 10 minutes before the spores in suspension were collected and adjusted to McFarland 0.5 (approximately 2×10⁶ CFU/mL) in sterile PBS. This was then diluted 1:50 in RPMI media to generate the inoculum, and 180 μL of the inoculum was combined with 20 μL of the 10× test article in a 96-well plate for a final concentration of 1×.

C. albicans colonies were collected with a sterile swab and used to make a McFarland 0.5 standard solution (approximately 1×10⁶ CFU/mL) in sterile PBS, then diluted 1:180 in RPMI media to generate the inoculum. 90 μL of the inoculum was then combined with 10 μL of the 10× test article in a 96-well plate for a final concentration of 1×. All test conditions were performed in duplicates.

C. albicans MIC 96-well plate was incubated for 37° C. for 24 to 48 hours. A. brasiliensis MIC 96-well plate was incubated at 25° C. for up to 7 days, until growth in control wells were observed. After incubation, all 96-well plates were examined visually and on a spectrophotometer at absorbance OD₆₅₀. MIC is defined as the lowest concentration of a test article in which no visible growth is observed. Results are presented in Tables 6 and 7A.

Minimum fungicidal concentration (MFC) was determined by plating 10 microliters of liquid from each MFC test solution on the appropriate agar media for each test strain. The liquid was allowed to air dry in a biosafety cabinet, then the agar plates were incubated under the appropriate conditions: A. brasiliensis and C. albicans were grown on Sabouraud Dextrose Agar (SDA) plates. A. brasiliensis was grown at 25° C. for 5 to 7 days, while C. albicans was grown at 37° C. for 24 to 48 hours. Colony formation was then assessed. MFC is defined as the lowest concentration of the Tgase (SEQ ID NO:6) in which no colonies were recovered. Results are presented in Table 7B.

TABLE 6
MIC values of chemical preservatives.

| | | MIC (μg/mL) | | | |
|---|---|---|---|---|---|
| | | | | Benzalkonium | |
| Species | Strain ID | Sodium benzoate | | Chloride | |
| S. aureus | ATCC 6538 | 10,000 | 10,000 | 1.95 | ≤0.98 |
| P. aeruginosa | ATCC 9027 | 10,000 | 10,000 | 62.5 | 31.25 |
| E. coli | ATCC 8739 | 10,000 | 10,000 | 15.63 | 15.63 |
| C. albicans | ATCC 10231 | >20,000 | >20,000 | 3.91 | 3.91 |
| A. brasilliensis | ATCC 16404 | 2,500 | 1,250 | 3.91 | 1.95 |

TABLE 7A
MIC values of Tgase variant (SEQ ID NO: 6)

| | | |
|---|---|---|
| | | MIC |
| Species | Strain ID | (μg/mL) |

| | | | |
|---|---|---|---|
| B. subtilis | BGSC 1A1276 | 80 | 120 |
| E. coli | ATCC 8739 | >1,000 | >1,000 |
| P. aeruginosa | ATCC 9027 | >1,000 | >1,000 |
| S. aureus | ATCC 6538 | >1,000 | >1,000 |

TABLE 7B
MFC values of Tgase variant (SEQ ID NO: 6)

| | | MIC | | MFC | |
|---|---|---|---|---|---|
| Species | Strain ID | (μg/mL) | | (μg/mL) | |
| A. brasilliensis | ATCC 16404 | 62.5 | 62.5 | 125 | 125 |
| C. albicans | ATCC 10231 | 15.6 | 31.25 | 31.25 | 62.5 |
| C. parapsilosis | ATCC 22019 | 250 | 125 | 250 | 125 |

### Example 4. Compatibility of Tgase with Chemical Preservatives

Using MIC values calculated previously (See Table 6), percent reduction in growth of E. coli in the presence of common chemical preservatives was measured (Table 8). A microplate assay of antimicrobial combinations was executed to determine if the presence of Tgase at different concentrations reduces efficacy of the preservative.

E. coli ATCC 8739 was grown overnight in LB broth at 37° C. The following day, the cell density of the saturated cultures was calculated using OD₆₀₀ and cultures were diluted to 10⁴ to 10⁶ CFU/mL in sterile LB media to generate the inoculum, and 90 μL of the inoculum was combined with 10 μL of serially diluted Tgase SEQ ID NO:6 at a range of 0.001-0.1 weight percent in the presence or absence of chemical preservatives at concentrations above and below the calculated MIC value. A single concentration (at the MIC) of the chemical preservative and static concentrations of Tgase SEQ ID NO:6 (400 μg/mL, 0.04% w/v) are presented in Table 8 as representative examples. Growth curves were measured by OD₆₀₀ on a BioTek® Synergy Plate Reader and a cell viability assay (BacTiter-Glo™, Promega®, following manufacturer's protocols) was used to assess cell viability. A decrease in OD₆₀₀ or luminescence indicated a decrease in cell viability. Results are presented as a percent reduction in cell count relative to an untreated culture. Addition of Tgase did not reduce the efficacy of the preservative.

TABLE 8
MIC values and growth inhibition of E. coli ATCC 8739

| | (μg/mL) | | | | | | |
|---|---|---|---|---|---|---|---|
| | | | | | | Tgase | Tgase |
| | sodium | | | 1,2- | 1,2- | SEQ ID | SEQ ID |
| | benzoate | BZK | MIT | octandiol | hexandiol | NO: 6 | NO: 1 |
| Concentration | 10,000 | 16 | 150 | 500 | 3,000 | 950 | 960 |
| growth inhibition | 99.85% | 99.7% | 98.7% | 97.8% | 97.9% | 85.0 | 0.0 |
| growth inhibition* | 99.97% | 99.94% | 99.8% | 99.3% | 99.5% | N/A | N/A |

*In the presence of 400 (μg/mL) Tgase SEQ ID NO: 6

Similar experiments are carried out for S. aureus ATCC 6538, P. aeruginosa ATCC 9027, C. albicans ATCC 10231, and A. brasiliensis ATCC 16404 using culture conditions described previously. These strains are diluted to 10⁴ to 10⁶ CFU/mL in sterile media to generate the inoculum, and 90 μL of the inoculum is combined with 10 μL of serially diluted Tgase SEQ ID NO:6 at a range of 0.001-0.1 weight percent in the presence or absence of chemical preservatives at concentrations above and below the calculated MIC value. Growth curves are measured by OD₆₀₀ on a BioTek® Synergy Plate Reader. Optionally, the following day, a cell viability assay such as BacTiter-Glo™ (Promega®, following manufacturer's protocols) can be used to assess cell viability. A decrease in OD₆₀₀ or luminescence indicates a decrease in cell viability. All test conditions are performed in triplicate to demonstrate Tgase efficacy against yeast and mold is maintained while preservative efficacy against bacterial strains are also maintained.

### Example 5. Additive Antimicrobial Behavior of Chitosan and Tgase SEQ ID NO:6

E. coli ATCC 8739 was grown overnight in LB broth at 37° C. The following day, the cell density of the saturated cultures was calculated using OD₆₀₀ and cultures were diluted to 10⁴ to 10⁶ CFU/mL in sterile LB media to generate the inoculum, and 90 μL of the inoculum was combined with 10 μL of serially diluted Tgase SEQ ID NO:6 at a concentration of 0.044% w/v (440 μg/mL) in the presence or absence of chitosan (250 μg/mL or 0.025% w/v). Growth curves were measured by OD₆₀₀ over 16 hours on a BioTek® Synergy Plate Reader, and a cell viability assay (BacTiter-Glo™, Promega®, following manufacturer's protocols) was used to assess cell viability. A decrease in OD₆₀₀ or luminescence indicated a decrease in cell viability. Results are presented as a percent reduction in cell count, relative to an untreated culture, in Table 9.

TABLE 9
MIC values and growth inhibition of E. coli ATCC 8739

| | | Tgase SEQ | |
|---|---|---|---|
| | Chitosan | ID NO: 6 | Chitosan:Tgase |
| Concentration | 250 | 950 | 125:220 |
| (μg/mL) | | | |
| growth inhibition | 86.9% | 85.0% | 99.7% |

### Example 6. Co-Formulation of Zymogen and Protease for Antimicrobial Activity

E. coli strain BL21(DE3) was purchased from New England Biolabs (Ipswich, MA), and transformed to produce S. mobaraensis pro-Tgase Variant SEQ ID NO:3 using standard methods known in the art. The transformed cells were cultured by shaking at 30-34° C. for up to 10 hours in a medium containing 10% glycerol, 0.75% soy peptone, 0.75% yeast extract, 0.5% magnesium sulfate heptahydrate, and 0.15% potassium phosphate monobasic. The culture was then induced with 0.1-0.4 mM isopropyl β-d-1-thiogalactopyranoside (IPTG) and incubated with agitation at 20-25° C. for up to 24 hours. The culture was centrifuged at 8000×g for up to 60 minutes. The supernatant was discarded, and the pellet was resuspended to 20% w/v in 50 mM tris(hydroxymethyl)aminomethane (Tris), 1 mM phenylmethylsulfonyl fluoride (PMSF), pH 8.

The cells were lysed using a high-pressure homogenizer at pressures from 15000-20000 psi. The crude lysates were clarified through centrifugation at 15000×g for up to 60 minutes. The clarified lysate containing pro-Tgase SEQ ID NO:3 was evaluated by sodium dodecyl sulfate polyacrylamide gel electrophoresis (SDS-PAGE) and spectroscopy (A280 nm). The clarified lysate was further purified by affinity column on a Ni-IMAC resin and desalted. Pro-Tgase SEQ ID NO:3 activity was measured using a colorimetric hydroxamate activity assay (Folk and Cole (1965) J Biol Chemistry 240(7):2951-2960) and revealed little to no activity of the pro-Tgase.

Genes encoding the wild-type S. mobaraensis Tgase proteases, transglutaminase activating metalloprotease (TAMEP) SEQ ID NO:4 and S. mobaraensis tripeptidyl aminopeptidase (SM-TAP) SEQ ID NO:5, were synthesized by Integrated DNA Technologies (Coralville, IA). Expression constructs for TAMEP and SM-TAP were designed with an N-terminal SacB signal sequence and hexa-His tag, and cloned using methods well known in the art. B. subtilis SCK6 delta-AlaR (purchased from BioTechnical Resources (Manitowoc, WI)) were grown overnight at 37° C. in 5 mL of LB medium supplemented with 40 mg/mL D-alanine. The following day, the culture was diluted to an OD₆₀₀ of 1.0 and xylose was added to a final concentration of 1%. After 2 hours, 250 μL of glycerol and ligated DNA was added and the culture tube was returned to the incubator for an additional 90 minutes. Following incubation, 10-1000 μL of culture was spread onto LB agar plates. Plates were grown at 37° C. overnight. The following day, 2-8 colonies were selected from each plate and inoculated into 3 mL of LB broth. Cultures were incubated at 37° C. for 48 hours and supernatant samples were taken periodically. SDS-PAGE was used to confirm secretion of the active form of the enzyme into the media as determined by molecular weight.

Activity was confirmed using protease activity assays well known in the art. Both proteases, TAMEP and SM-TAP, were isolated from their respective cell cultures by centrifugate at 8000×g for 10 minutes. The supernatant was used as isolated without further purification. Optionally, TAMEP and SM-TAP are purified by affinity column on a Ni-IMAC resin and desalted prior to use.

The antimicrobial properties of pro-Tgase SEQ ID NO:3 in combination with proteases TAMEP and SM-TAP are evaluated by creating a matrix in a 96-well plate where the concentration of the protease (0.0001-0.1% w/v, using total protein concentration of the expression media) and zymogen (0.001-1% w/v) are varied across the plate. MIC against bacterial strains (S. aureus ATCC 6538, E. coli ATCC 8739, P. aeruginosa ATCC 9027) and fungal strains, C. albicans ATCC 10231, and A. brasiliensis ATCC 16404 are determined using protocols described in Example 3. Antimicrobial properties are evaluated by the lowest concentration of zymogen (pro-Tgase variant SEQ ID NO:3) in the presence of the two proteases in which visible reduction of growth is observed on a spectrophotometer at absorbance OD₆₅₀.

### Example 7. Antimicrobial Properties of Tyrosinase

Commercially available wild-type mushroom tyrosinase (T3824, lyophilized powder, >1000 unit/mg solid) was purchased from Sigma-Aldrich and is used as received. The antimicrobial properties of tyrosinase are evaluated by treating S. aureus ATCC 6538, E. coli ATCC 8739, P. aeruginosa ATCC 9027, C. albicans ATCC 10231, and A. brasiliensis ATCC 16404 with tyrosinase (100-10,000 U) under the culture conditions described in Example 3. Antimicrobial properties are evaluated by reduction in visible growth of the bacterial or fungal strain on a spectrophotometer at absorbance OD₆₅₀.

### Example 8. Antimicrobial Properties of Lysyl Oxidase

Commercially available recombinant human lysyl oxidase (LOX-608H, 1 g/L buffered solution) was purchased from Creative Biomart (Shirley, NY) and is used as received. The antimicrobial properties of lysyl oxidase is evaluated by treating S. aureus ATCC 6538, E. coli ATCC 8739, P. aeruginosa ATCC 9027, C. albicans ATCC 10231, and A. brasiliensis ATCC 16404 with lysyl oxidase (0.0001-0.1% w/v) under the culture conditions described in Example 3. Antimicrobial properties are evaluated by reduction in visible growth of the bacterial or fungal strain on a spectrophotometer at absorbance OD₆₅₀.

### Example 9. Antimicrobial Properties of Laccase

Commercially available wild-type Aspergillus sp. laccase (SAE0050, liquid preparation) is purchased from Sigma-Aldrich and dialyzed prior to use to remove preservative in packaging. The antimicrobial properties of laccase are evaluated by treating S. aureus ATCC 6538, E. coli ATCC 8739, P. aeruginosa ATCC 9027, C. albicans ATCC 10231, and A. brasiliensis ATCC 16404 with laccase (0.0001-0.1% w/v) in the presence and absence of an initiator molecule such as 2, 2′-azino-bis-3-ethylbenzothiazoline-6-sulfonic acid (ABTS) (Sigma-Aldrich, 10102946001) (0.0001-0.001% w/v) under the culture conditions described in Example 3. Antimicrobial properties are evaluated by reduction in visible growth of the bacterial or fungal strain on a spectrophotometer at absorbance OD₆₅₀.

Amino Acid Sequences

SEQ ID NO: 1
Mature Streptomyces mobaraensis Tgase Wild Type
DSDDRVTPPAEPLDRMPDPYRPSYGRAETVVNNYIRKWQQVYSHRDGRKQQMTEEQRE
WLSYGCVGVTWVNSGQYPTNRLAFASFDEDRFKNELKNGRPRSGETRAEFEGRVAKESF
DEEKGFQRAREVASVMNRALENAHDESAYLDNLKKELANGNDALRNEDARSPFYSALR
NTPSFKERNGGNHDPSRMKAVIYSKHFWSGQDRSSSADKRKYGDPDAFRPAPGTGLVDM
SRDRNIPRSPTSPGEGFVNFDYGWFGAQTEADADKTVWTHGNHYHAPNGSLGAMHVYE
SKFRNWSEGYSDFDRGAYVITFIPKSWNTAPDKVKQGWP
SEQ ID NO: 2
Mature Streptomyces mobaraensis Tgase Variant
DPDDRVTPPAEPLDRMPDPYRPSYGRAETVVNNYIRKWQQVYSHRDGRKQQMTEEQRE
WLSYGCVGVTWVNSGQYPTNRLAFASFDEDRFKNELKNGRPRSGETRAEFEGRVAKESF
DEEKGFQRAREVASVMNRALENAHDESAYLDNLKKELANGNDALRNEDARSPFYSALR
NTPSFKERNGGNHDPSRMKAVIYSKHFWSGQDRSSSADKRKYGDPDAFRPAPGTGLVDM
SRDRNIPRSPTSPGEGFVNFDYGWFGAQTEADADKTVWTHGNHYHAPNGSLGAMHVYE
SKFRNWSEGYSDFDRGAYVITFIPKSWNTAPDKVKQGWP
SEQ ID NO: 3
Streptomyces mobaraensis Tgase zymogen Variant (pro-Tgase)
MDNGAGEETKSYAETYRLTADDVANINALNESAPAASSAGPSFRAPDPDDRVTPPAEPLD
RMPDPYRPSYGRAETVVNNYIRKWQQVYSHRDGRKQQMTEEQREWLSYGCVGVTWVN
SGQYPTNRLAFASFDEDRFKNELKNGRPRSGETRAEFEGRVAKESFDEEKGFQRAREVAS
VMNRALENAHDESAYLDNLKKELANGNDALRNEDARSPFYSALRNTPSFKERNGGNHD
PSRMKAVIYSKHFWSGQDRSSSADKRKYGDPDAFRPAPGTGLVDMSRDRNIPRSPTSPGE
GFVNFDYGWFGAQTEADADKTVWTHGNHYHAPNGSLGAMHVYESKFRNWSEGYSDFD
RGAYVITFIPKSWNTAPDKVKQGWPLEHHHHHH
SEQ ID NO: 4
Wild-type Streptomyces mobaraensis Pro-TAMEP (pro-TAMEP; UniProt
P83543)
GQDKAAHPAPRQSIHKPDPGAEPVKLTPSQRAELIRDANATKAETAKNLGLGAKEKLVV
KDVVKDKNGTLHTRYERTYDGLPVLGGDLVVDATRSGQVKTAAKATKQRIAVASTTPS
LAASAAEKDAVKAARAKGSKAGKADKAPRKVVWAAKGTPVLAYETVVGGVQDDGTPS
QLHVITDAKTGKKLFEFQGVKQGTGNSQHSGQVQIGTTKSGSSYQMNDTTRGGHKTYNL
NHGSSGTGTLFTDSDDVWGNGTNSDPATAGVDAHYGAQLTWDYYKNVHGRNGIRGDG
VGAYSRVHYGNNYVNAFWDDSCFCMTYGDGNGIPLTSIDVAAHEMTHGVTSATANLTY
SGESGGLNEATSDMMATAVEFWANNPADPGDYLIGEKININGDGTPLRYMDKPSKDGAS
KDAWYSGLGGIDVHYSSGPANHWFYLASEGSGPKDIGGVHYDSPTSDGLPVTGVGRDNA
AKIWFKALTERMQSNTDYKGARDATLWAAGELFGVNSDTYNNVANAWAAINVGPRAS
SGVSVTSPGDQTSIVNQAVSLQIKATGSTSGALTYSATGLPAGLSINASTGLISGTPTTTGTS
NVTVTVKDSAGKTGSTSFKWTVNTTGGGSVFENTTQVAIPDAGAAVTSPIVVTRSGNGPS
ALKVDVNITHTYRGDLTIDLVAPNGKTWRLKNSDAWDSAADVSETYTVDASSVSANGT
WKLKVQDVYSGDSGTIDKWRLTFHHHHHH
SEQ ID NO: 5 SM-TAP
Wild-type Streptomyces mobaraensis Pro- SM-TAP (pro-SM-TAP;
UniProt P83615)
ASITAPQADIKDRILKIPGMKFVEEKPYQGYRYLVMTYRQPVDHRNPGKGTFEQRFTLLH
KDTDRPTVFFTSGYNVSTNPSRSEPTRIVDGNQVSMEYRFFTPSRPQPADWSKLDIWQAA
SDQHRLYQALKPVYGKNWLATGGSKGGMTATYFRRFYPNDMNGTVAYVAPNDVNDKE
DSAYDKFFQNVGDKACRTQLNSVQREALVRRDEIVARYEKWAKENGKTFKVVGSADKA
YENVVLDLVWSFWQYHLQSDCASVPATKASTDELYKFIDDISGFDGYTDQGLERFTPYY
YQAGTQLGAPTVKNPHLKGVLRYPGINQPRSYVPRDIPMTFRPGAMADVDRWVREDSRN
MLFVYGQNDPWSGEPFRLGKGAAARHDYRFYAPGGNHGSNIAQLVADERAKATAEVLK
WAGVAPQAVQKDEKAAKPLAPFDAKLDRVKNDKQSALRPHHHHHH
SEQ ID NO: 6
Mature Streptomyces mobaraensis Tgase Variant (SEQ ID NO: 2
including N-terminal Methionine and C-terminal peptide linker and
Hex-His-Tag)
MDPDDRVTPPAEPLDRMPDPYRPSYGRAETVVNNYIRKWQQVYSHRDGRKQQMTEEQR
EWLSYGCVGVTWVNSGQYPTNRLAFASFDEDRFKNELKNGRPRSGETRAEFEGRVAKES
FDEEKGFQRAREVASVMNRALENAHDESAYLDNLKKELANGNDALRNEDARSPFYSAL
RNTPSFKERNGGNHDPSRMKAVIYSKHFWSGQDRSSSADKRKYGDPDAFRPAPGTGLVD
MSRDRNIPRSPTSPGEGFVNFDYGWFGAQTEADADKTVWTHGNHYHAPNGSLGAMHVY
ESKFRNWSEGYSDFDRGAYVITFIPKSWNTAPDKVKQGWPLEHHHHHH

What is claimed is:

1. A composition comprising (a) at least one crosslinking enzyme, optionally in the form of a zymogen, in combination with (b) at least one component selected from the group consisting of enzymes, peptides, and/or proteins, optionally having antimicrobial activity, and optionally further in combination with (c) at least one chemical preservative, wherein the composition comprising (a) in combination with (b), and optionally further in combination with (c), has at least one activity selected from the group consisting of preservative and antimicrobial.

2. The composition of claim 1 wherein the at least one crosslinking enzyme is selected from the group consisting of transglutaminases, lysyl oxidases, tyrosinases, laccases, sortases, formylglycine-generating enzymes, and sulfhydryl oxidases.

3. The composition of claim 1 or 2 wherein the at least one crosslinking enzyme is a transglutaminase.

4. The composition of claim 1 or 2 wherein the at least one crosslinking enzyme has at least 90% sequence identity with the amino acid sequence set forth in SEQ ID NO:2.

5. The composition of claim 3 wherein the at least one crosslinking enzyme has at least 90% sequence identity with the amino acid sequence set forth in SEQ ID NO:2.

6. The composition of claim 1, 2 or 5 wherein the at least one component having antimicrobial activity is selected from the group consisting of lysozyme, chitinase, lipase, lysin, lysostaphin, glucanase, DNase, RNase, lactoferrin, glucose oxidase, peroxidase, lactoperoxidase, lactonase, acylase, dispersin B, amylases, proteases, cellulase, nisin, bacteriocin, siderophore, polymyxin, and defensin.

7. The composition of claim 3 wherein the at least one component having antimicrobial activity is selected from the group consisting of lysozyme, chitinase, lipase, lysin, lysostaphin, glucanase, DNase, RNase, lactoferrin, glucose oxidase, peroxidase, lactoperoxidase, lactonase, acylase, dispersin B, amylases, proteases, cellulase, nisin, bacteriocin, siderophore, polymyxin, and defensin.

8. The composition of claim 4 wherein the at least one component having antimicrobial activity is selected from the group consisting of lysozyme, chitinase, lipase, lysin, lysostaphin, glucanase, DNase, RNase, lactoferrin, glucose oxidase, peroxidase, lactoperoxidase, lactonase, acylase, dispersin B, amylases, proteases, cellulase, nisin, bacteriocin, siderophore, polymyxin, and defensin.

9. The composition of claim 1, 2 or 5 wherein the at least one chemical preservative is selected from the group consisting of quaternary ammonium compounds, detergents, chaotropic agents, organic acids, alcohols, glycols, aldehydes, oxidizers, parabens, isothiazolinones, and cationic polymers.

10. The composition of claim 3 wherein the at least one chemical preservative is selected from the group consisting of quaternary ammonium compounds, detergents, chaotropic agents, organic acids, alcohols, glycols, aldehydes, oxidizers, parabens, isothiazolinones, and cationic polymers.

11. The composition of claim 4 wherein the at least one chemical preservative is selected from the group consisting of quaternary ammonium compounds, detergents, chaotropic agents, organic acids, alcohols, glycols, aldehydes, oxidizers, parabens, isothiazolinones, and cationic polymers.

12. The composition of claim 1 wherein (a) is a zymogen and (b) comprises an enzyme, further wherein the zymogen and the enzyme interact to produce an active enzyme having at least one activity selected from the group consisting of preservative and antimicrobial.

13. An expression vector comprising at least one heterologous nucleic acid sequence that encodes at least one crosslinking enzyme, optionally in the form of a zymogen, wherein said heterologous nucleic acid sequence is optionally operably linked to at least one regulatory sequence and wherein the expression vector is capable of transforming a host cell to express, either intracellularly or extracellularly, said at least one crosslinking enzyme so that the transformed host cell is inactivated, inhibited, or killed.

14. The expression vector of claim 13 wherein the at least one crosslinking enzyme is selected from the group consisting of transglutaminases, lysyl oxidases, tyrosinases, laccases, sortases, formylglycine-generating enzymes, and sulfhydryl oxidases.

15. The expression vector of claim 13 or 14 wherein the at least one crosslinking enzyme is a transglutaminase.

16. The expression vector of claim 13 or 14 wherein the at least one crosslinking enzyme has at least 90% sequence identity with the amino acid sequence set forth in SEQ ID NO:2.

17. The expression vector of claim 15 wherein the at least one crosslinking enzyme has at least 90% sequence identity with the amino acid sequence set forth in SEQ ID NO:2.


SEQUENCE LISTING (7 sequences)

SEQ ID NO: 1 - protein, 331 aa, Streptomyces mobaraensis
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 2 - protein, 331 aa, Artificial Sequence
  Description of Artificial Sequence Synthetic polypeptide
DPDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 3 - protein, 385 aa, Artificial Sequence
  Description of Artificial Sequence Synthetic polypeptide
MDNGAGEETKSYAETYRLTADDVANINALNESAPAASSAGPSFRAPDPDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWPLEHHHHHH

SEQ ID NO: 4 - protein, 733 aa, Artificial Sequence
  Description of Artificial Sequence Synthetic polypeptide
GQDKAAHPAPRQSIHKPDPGAEPVKLTPSQRAELIRDANATKAETAKNLGLGAKEKLVVKDVVKDKNGTLHTRYERTYDGLPVLGGDLVVDATRSGQVKTAAKATKQRIAVASTTPSLAASAAEKDAVKAARAKGSKAGKADKAPRKVVWAAKGTPVLAYETVVGGVQDDGTPSQLHVITDAKTGKKLFEFQGVKQGTGNSQHSGQVQIGTTKSGSSYQMNDTTRGGHKTYNLNHGSSGTGTLFTDSDDVWGNGTNSDPATAGVDAHYGAQLTWDYYKNVHGRNGIRGDGVGAYSRVHYGNNYVNAFWDDSCFCMTYGDGNGIPLTSIDVAAHEMTHGVTSATANLTYSGESGGLNEATSDMMATAVEFWANNPADPGDYLIGEKININGDGTPLRYMDKPSKDGASKDAWYSGLGGIDVHYSSGPANHWFYLASEGSGPKDIGGVHYDSPTSDGLPVTGVGRDNAAKIWFKALTERMQSNTDYKGARDATLWAAGELFGVNSDTYNNVANAWAAINVGPRASSGVSVTSPGDQTSIVNQAVSLQIKATGSTSGALTYSATGLPAGLSINASTGLISGTPTTTGTSNVTVTVKDSAGKTGSTSFKWTVNTTGGGSVFENTTQVAIPDAGAAVTSPIVVTRSGNGPSALKVDVNITHTYRGDLTIDLVAPNGKTWRLKNSDAWDSAADVSETYTVDASSVSANGTWKLKVQDVYSGDSGTIDKWRLTFHHHHHH

SEQ ID NO: 5 - protein, 456 aa, Artificial Sequence
  Description of Artificial Sequence Synthetic polypeptide
ASITAPQADIKDRILKIPGMKFVEEKPYQGYRYLVMTYRQPVDHRNPGKGTFEQRFTLLHKDTDRPTVFFTSGYNVSTNPSRSEPTRIVDGNQVSMEYRFFTPSRPQPADWSKLDIWQAASDQHRLYQALKPVYGKNWLATGGSKGGMTATYFRRFYPNDMNGTVAYVAPNDVNDKEDSAYDKFFQNVGDKACRTQLNSVQREALVRRDEIVARYEKWAKENGKTFKVVGSADKAYENVVLDLVWSFWQYHLQSDCASVPATKASTDELYKFIDDISGFDGYTDQGLERFTPYYYQAGTQLGAPTVKNPHLKGVLRYPGINQPRSYVPRDIPMTFRPGAMADVDRWVREDSRNMLFVYGQNDPWSGEPFRLGKGAAARHDYRFYAPGGNHGSNIAQLVADERAKATAEVLKWAGVAPQAVQKDEKAAKPLAPFDAKLDRVKNDKQSALRPHHHHHH

SEQ ID NO: 6 - protein, 340 aa, Artificial Sequence
  Description of Artificial Sequence Synthetic polypeptide
MDPDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWPLEHHHHHH

SEQ ID NO: 7 - protein, 6 aa, Artificial Sequence
  Description of Artificial Sequence Synthetic 6xHis tag
HHHHHH